(19) United States
(12) Patent Application Publication
(10) Pub. No.: US 2021/0161502 A1
(43) Pub. Date: Jun. 3, 2021

(54) SYSTEM AND METHOD FOR DETERMINING A LIKELIHOOD OF PARADOXICAL VOCAL CORD MOTION (PVCM) IN A PERSON

(71) Applicant: Samsung Electronics Co., Ltd., Suwon-si (KR)

(72) Inventors: Viswam Nathan, Mountain View, CA (US); Md Mahbubur Rahman, San Jose, CA (US); Ebrahim Nemati, Santa Clara, CA (US); Korosh Vatanparvar, Santa Clara, CA (US); Jilong Kuang, San Jose, CA (US); Jun Gao, Menlo Park, CA (US)

(21) Appl. No.: 16/702,313

(22) Filed: Dec. 3, 2019

(51) Int. Cl. A61B 7/00; G16H 50/50; G16H 50/20; G16H 40/67; A61B 5/08; A61B 5/00; A61B 7/04

(52) U.S. Cl. CPC A61B 7/003; G16H 50/50; G16H 50/20; G16H 40/67; A61B 2562/0204; A61B 5/742; A61B 5/7275; A61B 7/04; A61B 5/0022; A61B 5/0823

(57) ABSTRACT

A method includes receiving sensor data associated with respiration of a user at a first electronic device. The method also includes extracting features specific to paradoxical vocal cord motion (PVCM) from the sensor data, the extracted features comprising a cough wetness level and a respiration phase difficulty level. The method also includes calculating a PVCM score based on the extracted features using a predetermined model, wherein the predetermined model allocates weights to the extracted features based on respective importance to PVCM determination. The method also includes presenting an indicator on a display of the first electronic device for use by the user or a medical provider, the indicator representing the PVCM score.

### TECHNICAL FIELD

This disclosure relates generally to health monitoring systems and methods. More specifically, this disclosure relates to a system and method for determining a likelihood of paradoxical vocal cord motion (PVCM) in a person.

### BACKGROUND

Chronic respiratory diseases (chronic diseases of the airways) currently affect an estimated 40 million people in the United States alone. Common respiratory diseases include asthma, chronic obstructive pulmonary disease (COPD), occupational lung disease, and chronic bronchitis. Another respiratory condition that is symptomatically similar to asthma is paradoxical vocal cord motion (PVCM). PVCM is a condition in which the vocal cords close instead of open when breathing in, thus allowing less air to be inhaled. Many patients who have breathing difficulties are often misdiagnosed with asthma when they actually have PVCM. This condition is more psychological and does not respond to asthma medication. Diagnosis of PVCM can be difficult in a short time period, such as a one-time visit to a health care provider.

### SUMMARY

This disclosure provides a system and method for determining a likelihood of paradoxical vocal cord motion (PVCM) in a person.

In a first embodiment, a method includes receiving sensor data associated with respiration of a user at a first electronic device. The method also includes extracting features specific to PVCM from the sensor data, the extracted features comprising a cough wetness level and a respiration phase difficulty level. The method also includes calculating a PVCM score based on the extracted features using a predetermined model, wherein the predetermined model allocates weights to the extracted features based on respective importance to PVCM determination. The method also includes presenting an indicator on a display of the first electronic device for use by the user or a medical provider, the indicator representing the PVCM score.

In a second embodiment, an electronic device includes a display, a processor, and a memory coupled to the processor. The memory stores instructions executable by the processor to receive sensor data associated with respiration of a user; extract features specific to paradoxical vocal cord motion (PVCM) from the sensor data, the extracted features comprising a cough wetness level and a respiration phase difficulty level; calculate a PVCM score based on the extracted features using a predetermined model, wherein the predetermined model allocates weights to the extracted features based on respective importance to PVCM determination; and present an indicator on the display for use by the user or a medical provider, the indicator representing the PVCM score.

In a third embodiment, a non-transitory computer readable medium contains computer readable program code that, when executed, causes at least one processor to receive sensor data associated with respiration of a user; extract features specific to paradoxical vocal cord motion (PVCM) from the sensor data, the extracted features comprising a cough wetness level and a respiration phase difficulty level; calculate a PVCM score based on the extracted features using a predetermined model, wherein the predetermined model allocates weights to the extracted features based on respective importance to PVCM determination; and present an indicator on a display for use by the user or a medical provider, the indicator representing the PVCM score.

Other technical features may be readily apparent to one skilled in the art from the following figures, descriptions, and claims.

Before undertaking the DETAILED DESCRIPTION below, it may be advantageous to set forth definitions of certain words and phrases used throughout this patent document. The terms “transmit,” “receive,” and “communicate,” as well as derivatives thereof, encompass both direct and indirect communication. The terms “include” and “comprise,” as well as derivatives thereof, mean inclusion without limitation. The term “or” is inclusive, meaning and/or. The phrase “associated with,” as well as derivatives thereof, means to include, be included within, interconnect with, contain, be contained within, connect to or with, couple to or with, be communicable with, cooperate with, interleave, juxtapose, be proximate to, be bound to or with, have, have a property of, have a relationship to or with, or the like.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

As used here, terms and phrases such as “have,” “may have,” “include,” or “may include” a feature (like a number, function, operation, or component such as a part) indicate the existence of the feature and do not exclude the existence of other features. Also, as used here, the phrases “A or B,” “at least one of A and/or B,” or “one or more of A and/or B” may include all possible combinations of A and B. For example, “A or B,” “at least one of A and B,” and “at least one of A or B” may indicate all of (1) including at least one A, (2) including at least one B, or (3) including at least one A and at least one B.

As used here, the terms “first” and “second” may modify various components regardless of importance and do not limit the components. These terms are only used to distinguish one component from another. For example, a first user device and a second user device may indicate different user devices from each other, regardless of the order or importance of the devices. A first component may be denoted a second component and vice versa without departing from the scope of this disclosure.

It will be understood that, when an element (such as a first element) is referred to as being (operatively or communicatively) “coupled with/to” or “connected with/to” another element (such as a second element), it can be coupled or connected with/to the other element directly or via a third element. In contrast, it will be understood that, when an element (such as a first element) is referred to as being “directly coupled with/to” or “directly connected with/to” another element (such as a second element), no other element (such as a third element) intervenes between the element and the other element.

As used here, the phrase “configured (or set) to” may be interchangeably used with the phrases “suitable for,” “having the capacity to,” “designed to,” “adapted to,” “made to,” or “capable of” depending on the circumstances. The phrase “configured (or set) to” does not essentially mean “specifically designed in hardware to.” Rather, the phrase “configured to” may mean that a device can perform an operation together with another device or parts. For example, the phrase “processor configured (or set) to perform A, B, and C” may mean a generic-purpose processor (such as a CPU or application processor) that may perform the operations by executing one or more software programs stored in a memory device or a dedicated processor (such as an embedded processor) for performing the operations.

The terms and phrases as used here are provided merely to describe some embodiments of this disclosure but not to limit the scope of other embodiments of this disclosure. It is to be understood that the singular forms “a,” “an,” and “the” include plural references unless the context clearly dictates otherwise. All terms and phrases, including technical and scientific terms and phrases, used here have the same meanings as commonly understood by one of ordinary skill in the art to which the embodiments of this disclosure belong. It will be further understood that terms and phrases, such as those defined in commonly-used dictionaries, should be interpreted as having a meaning that is consistent with their meaning in the context of the relevant art and will not be interpreted in an idealized or overly formal sense unless expressly so defined here. In some cases, the terms and phrases defined here may be interpreted to exclude embodiments of this disclosure.

Examples of an “electronic device” according to embodiments of this disclosure may include at least one of a smart phone, a tablet personal computer (PC), a mobile phone, a video phone, an e-book reader, a desktop PC, a laptop computer, a netbook computer, a workstation, a personal digital assistant (PDA), a portable multimedia player (PMP), an MP3 player, a mobile medical device, a camera, or a wearable device (such as smart glasses, a head-mounted device (HMD), electronic clothes, an electronic bracelet, an electronic necklace, an electronic appcessory, an electronic tattoo, a smart mirror, or a smart watch). Other examples of an electronic device include a smart home appliance. Examples of the smart home appliance may include at least one of a television, a digital video disc (DVD) player, an audio player, a refrigerator, an air conditioner, a cleaner, an oven, a microwave oven, a washer, a drier, an air cleaner, a set-top box, a home automation control panel, a security control panel, a TV box (such SAMSUNG HOMESYNC, APPLETV, or GOOGLE TV), a gaming console (such as an XBOX, PLAYSTATION, or NINTENDO), an electronic dictionary, an electronic key, a camcorder, or an electronic picture frame. Still other examples of an electronic device include at least one of various medical devices (such as diverse portable medical measuring devices (like a blood sugar measuring device, a heartbeat measuring device, or a body temperature measuring device), a magnetic resource angiography (MRA) device, a magnetic resource imaging (MRI) device, a computed tomography (CT) device, an imaging device, or an ultrasonic device), a navigation device, a global positioning system (GPS) receiver, an event data recorder (EDR), a flight data recorder (FDR), an automotive infotainment device, a sailing electronic device (such as a sailing navigation device or a gyro compass), avionics, security devices, vehicular head units, industrial or home robots, automatic teller machines (ATMs), point of sales (POS) devices, or Internet of Things (IoT) devices (such as a bulb, various sensors, electric or gas meter, sprinkler, fire alarm, thermostat, street light, toaster, fitness equipment, hot water tank, heater, or boiler). Other examples of an electronic device include at least one part of a piece of furniture or building/structure, an electronic board, an electronic signature receiving device, a projector, or various measurement devices (such as devices for measuring water, electricity, gas, or electromagnetic waves). Note that, according to embodiments of this disclosure, an electronic device may be one or a combination of the above-listed devices. According to some embodiments of this disclosure, the electronic device may be a flexible electronic device. The electronic device disclosed here is not limited to the above-listed devices and may include new electronic devices depending on the development of technology.

In the following description, electronic devices are described with reference to the accompanying drawings, according to embodiments of this disclosure. As used here, the term “user” may denote a human or another device (such as an artificial intelligent electronic device) using the electronic device.

Definitions for other certain words and phrases may be provided throughout this patent document. Those of ordinary skill in the art should understand that in many if not most instances, such definitions apply to prior as well as future uses of such defined words and phrases.

None of the description in this application should be read as implying that any particular element, step, or function is an essential element that must be included in the claim scope. The scope of patented subject matter is defined only by the claims. Moreover, none of the claims is intended to invoke 35 U.S.C. § 112(f) unless the exact words “means for” are followed by a participle. Use of any other term, including without limitation “mechanism,” “module,” “device,” “unit,” “component,” “element,” “member,” “apparatus,” “machine,” “system,” “processor,” or “controller,” within a claim is understood by the Applicant to refer to structures known to those skilled in the relevant art and is not intended to invoke 35 U.S.C. § 112(f).

### BRIEF DESCRIPTION OF THE DRAWINGS

For a more complete understanding of this disclosure and its advantages, reference is now made to the following description, taken in conjunction with the accompanying drawings, in which:

FIG. 1 illustrates an example network configuration in accordance with this disclosure;

FIG. 2 illustrates an example electronic device in accordance with this disclosure;

FIG. 3 illustrates an example process for determining a likelihood of paradoxical vocal cord motion (PVCM) in a person in accordance with this disclosure;

FIG. 4 illustrates an example system for determining a likelihood of PVCM in a person in accordance with this disclosure;

FIGS. 5 through 7 illustrate other example systems for determining a likelihood of PVCM in a person in accordance with this disclosure; and

FIG. 8 illustrates an example method for determining a likelihood of PVCM in a person in accordance with this disclosure.

### DETAILED DESCRIPTION

The figures discussed below and the various embodiments used to describe the principles of this disclosure in this patent document are by way of illustration only and should not be construed in any way to limit the scope of the disclosure. Those skilled in the art will understand that the principles of this disclosure can be implemented in any suitably arranged system.

As discussed above, paradoxical vocal cord motion (PVCM) is a condition in which the vocal cords close instead of open when breathing in, thus allowing less air to be inhaled. The symptoms of this condition can be very similar to those of asthma. It is estimated that 3% to 27% of the more than 25 million asthma patients in the United States may be misdiagnosed with PVCM. This is a concern because it can be dangerous to perform asthma treatments, perform invasive procedures, and prescribe asthma medication for patients who do not have asthma. Moreover, patients with undiagnosed PVCM can have considerably more visits to health care providers, and have higher medical utilization, than accurately diagnosed patients. Therefore, accurate diagnosis of PVCM can have economic benefits for the patient and ease the burden on clinicians.

Current methods for a positive diagnosis of PVCM include the use of laryngoscopy. This is an invasive procedure that involves inserting a camera via a tube into the oral cavity. There are risks and costs associated with this procedure; thus, it is not recommended for general screening practice. Moreover, for laryngoscopy to be successful, the patient has to have a PVCM attack while the tube is inserted. For example, studies have shown that laryngoscopy is very effective in identifying symptomatic PVCM patients, but only 55 to 60% effective for patients who are asymptomatic. This means that even laryngoscopy does not guarantee a definitive diagnosis if the patient fails to have an attack at the time of the procedure.

To address these and other issues, embodiments of this disclosure provide systems and methods for determining a likelihood of PVCM and differentiating between PVCM and asthma using one or more mobile sensors. The disclosed embodiments use long-term monitoring with wearable or mobile devices, such as a smart phone or smart watch, to extract PVCM-specific features (e.g., cough wetness, respiration phase difficulty, and the like) from symptoms exhibited by the user. The manifestation of symptoms of PVCM and the surrounding contexts are subtly different from those of asthma, and these symptoms can be observed through regular monitoring over a period of time. The observed symptoms can then be used for determining the likelihood of PVCM. For example, the observed symptoms can be used in a scenario where there is a doubt about whether PVCM or asthma is currently manifesting in the user.

The disclosed systems are automated and can be mostly or fully passive, requiring few or no actions by a user. The disclosed embodiments detect events, including pulmonary events of the user, and generate or update a PVCM likelihood score for the user. The disclosed score is generated by taking into account the user's risk factors (e.g., age, previous asthma diagnosis, etc.). The score is generated based on a model that ranks the features; the model can be updated dynamically based on observations from the sensors. If the score is sufficiently high, the user is notified of possible PVCM. A report can be generated for the primary symptoms and contexts for PVCM attacks. Upon a visit to a health care provider, this report can be used by the provider to direct treatment. In some instances, this report can improve laryngoscopic diagnostic rates for asymptomatic PVCM patients.

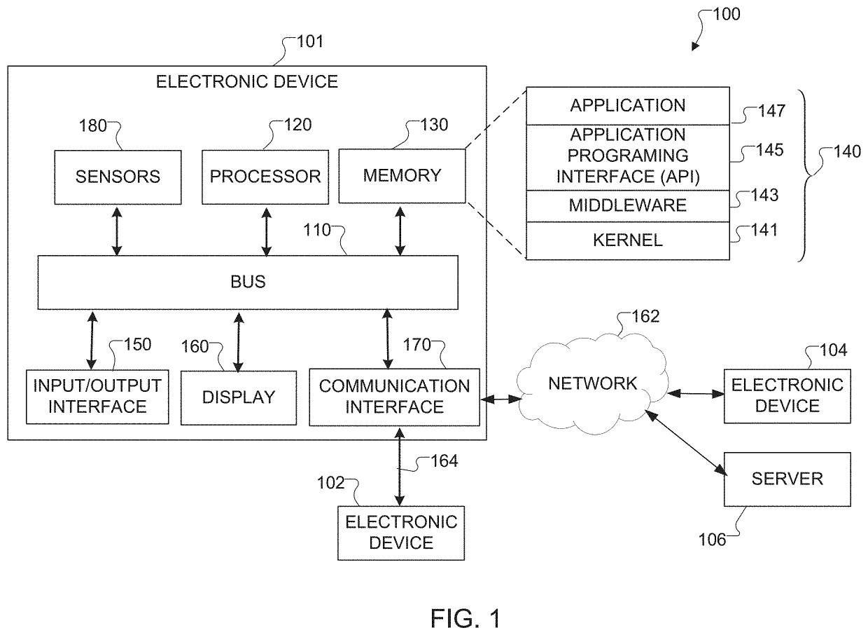

FIG. 1 illustrates an example network configuration 100 in accordance with this disclosure. As shown in FIG. 1, according to embodiments of this disclosure, an electronic device 101 is included in the network configuration 100. The electronic device 101 may include at least one of a bus 110, a processor 120, a memory 130, an input/output (I/O) interface 150, a display 160, a communication interface 170, or a sensor 180. In some embodiments, the electronic device 101 may exclude at least one of the components or may add another component.

The bus 110 may include a circuit for connecting the components 120-180 with one another and transferring communications (such as control messages and/or data) between the components. The processor 120 may include one or more of a central processing unit (CPU), an application processor (AP), or a communication processor (CP). The processor 120 may perform control on at least one of the other components of the electronic device 101 and/or perform an operation or data processing relating to communication.

The memory 130 may include a volatile and/or non-volatile memory. For example, the memory 130 may store commands or data related to at least one other component of the electronic device 101. According to embodiments of this disclosure, the memory 130 may store software and/or a program 140. The program 140 may include, for example, a kernel 141, middleware 143, an application programming interface (API) 145, and/or an application program (or “application”) 147. At least a portion of the kernel 141, middleware 143, or API 145 may be denoted an operating system (OS).

The kernel 141 may control or manage system resources (such as the bus 110, processor 120, or memory 130) used to perform operations or functions implemented in other programs (such as the middleware 143, API 145, or application program 147). The kernel 141 may provide an interface that allows the middleware 143, API 145, or application 147 to access the individual components of the electronic device 101 to control or manage the system resources. The middleware 143 may function as a relay to allow the API 145 or the application 147 to communicate data with the kernel 141, for example. A plurality of applications 147 may be provided. The middleware 143 may control work requests received from the applications 147, such as by allocating the priority of using the system resources of the electronic device 101 (such as the bus 110, processor 120, or memory 130) to at least one of the plurality of applications 147. The API 145 is an interface allowing the application 147 to control functions provided from the kernel 141 or the middleware 143. For example, the API 133 may include at least one interface or function (such as a command) for file control, window control, image processing, or text control.

The input/output interface 150 may serve as an interface that may, for example, transfer commands or data input from a user or other external devices to other component(s) of the electronic device 101. Further, the input/output interface 150 may output commands or data received from other component(s) of the electronic device 101 to the user or the other external devices.

The display 160 may include, for example, a liquid crystal display (LCD), a light emitting diode (LED) display, an organic light emitting diode (OLED) display, an active matrix OLED (AMOLED), a microelectromechanical systems (MEMS) display, or an electronic paper display. The display 160 can also be a depth-aware display, such as a multi-focal display. The display 160 may display various contents (such as text, images, videos, icons, or symbols) to the user. The display 160 may include a touchscreen and may receive, for example, a touch, gesture, proximity, or hovering input using an electronic pen or a body portion of the user.

The communication interface 170 may set up communication between the electronic device 101 and an external electronic device (such as a first electronic device 102, a second electronic device 104, or a server 106). For example, the communication interface 170 may be connected with a network 162 or 164 through wireless or wired communication to communicate with the external electronic device.

The electronic device 101 further includes one or more sensors 180 that can meter a physical quantity or detect an activation state of the electronic device 101 and convert metered or detected information into an electrical signal. For example, one or more sensors 180 can include one or more buttons for touch input, one or more cameras, a gesture sensor, a gyroscope or gyro sensor, an air pressure sensor, a magnetic sensor or magnetometer, an acceleration sensor or accelerometer, a grip sensor, a proximity sensor, a color sensor (such as a red green blue (RGB) sensor), a bio-physical sensor, a temperature sensor, a humidity sensor, an illumination sensor, an ultraviolet (UV) sensor, an electromyography (EMG) sensor, an electroencephalogram (EEG) sensor, an electrocardiogram (ECG) sensor, an infrared (IR) sensor, an ultrasound sensor, an iris sensor, or a fingerprint sensor. The sensor(s) 180 can also include an inertial measurement unit, which can include one or more accelerometers, gyroscopes, and other components. The sensor(s) 180 can further include a control circuit for controlling at least one of the sensors included here. Any of these sensor(s) 180 can be located within the electronic device 101.

The first external electronic device 102 or the second external electronic device 104 may be a wearable device or an electronic device 101-mountable wearable device (such as a head mounted display (HMD)). When the electronic device 101 is mounted in an HMD (such as the electronic device 102), the electronic device 101 may detect the mounting in the HMD and operate in a virtual reality mode. When the electronic device 101 is mounted in the electronic device 102 (such as the HMD), the electronic device 101 may communicate with the electronic device 102 through the communication interface 170. The electronic device 101 may be directly connected with the electronic device 102 to communicate with the electronic device 102 without involving with a separate network.

The wireless communication may use at least one of, for example, long term evolution (LTE), long term evolution-advanced (LTE-A), code division multiple access (CDMA), wideband code division multiple access (WCDMA), universal mobile telecommunication system (UMTS), wireless broadband (WiBro), or global system for mobile communication (GSM), as a cellular communication protocol. The wired connection may include at least one of, for example, universal serial bus (USB), high definition multimedia interface (HDMI), recommended standard 232 (RS-232), or plain old telephone service (POTS). The network 162 may include at least one communication network, such as a computer network (like a local area network (LAN) or wide area network (WAN)), the Internet, or a telephone network.

The first and second external electronic devices 102 and 104 each may be a device of the same type or a different type from the electronic device 101. According to embodiments of this disclosure, the server 106 may include a group of one or more servers. Also, according to embodiments of this disclosure, all or some of the operations executed on the electronic device 101 may be executed on another or multiple other electronic devices (such as the electronic devices 102 and 104 or server 106). Further, according to embodiments of this disclosure, when the electronic device 101 should perform some function or service automatically or at a request, the electronic device 101, instead of executing the function or service on its own or additionally, may request another device (such as electronic devices 102 and 104 or server 106) to perform at least some functions associated therewith. The other electronic device (such as electronic devices 102 and 104 or server 106) may execute the requested functions or additional functions and transfer a result of the execution to the electronic device 101. The electronic device 101 may provide a requested function or service by processing the received result as it is or additionally. To that end, a cloud computing, distributed computing, or client-server computing technique may be used, for example.

While FIG. 1 shows that the electronic device 101 includes the communication interface 170 to communicate with the external electronic device 102 or 104 or server 106 via the network(s) 162 and 164, the electronic device 101 may be independently operated without a separate communication function, according to embodiments of this disclosure. Also, note that the electronic device 102 or 104 or the server 106 could be implemented using a bus, a processor, a memory, an I/O interface, a display, a communication interface, and an event processing module (or any suitable subset thereof) in the same or similar manner as shown for the electronic device 101.

Although FIG. 1 illustrates one example of a network configuration 100, various changes may be made to FIG. 1. For example, the network configuration 100 could include any number of each component in any suitable arrangement. In general, computing and communication systems come in a wide variety of configurations, and FIG. 1 does not limit the scope of this disclosure to any particular configuration. Also, while FIG. 1 illustrates one operational environment in which various features disclosed in this patent document can be used, these features could be used in any other suitable system.

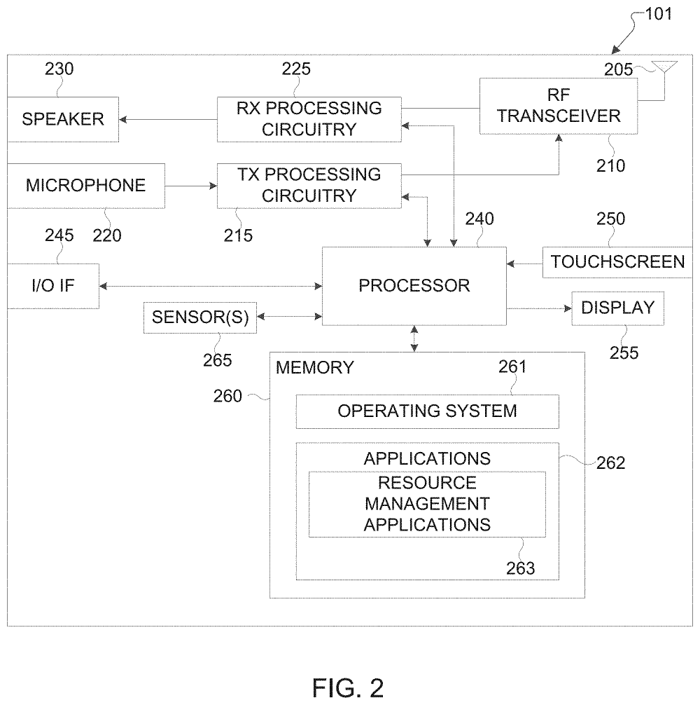

FIG. 2 illustrates an example electronic device 101 in accordance with this disclosure. The electronic device 101 could represent one or more of the electronic devices 101, 102, or 104 in FIG. 1. As shown in FIG. 2, the electronic device 101 includes an antenna 205, a radio frequency (RF) transceiver 210, transmit (TX) processing circuitry 215, a microphone 220, and receive (RX) processing circuitry 225. The electronic device 101 also includes a speaker 230, a processor 240, an input/output (I/O) interface (IF) 245, an input 250, a display 255, and a memory 260. The memory 260 includes an operating system (OS) program 261 and one or more applications 262.

The RF transceiver 210 receives, from the antenna 205, an incoming RF signal transmitted by another component in a system. The RF transceiver 210 down-converts the incoming RF signal to generate an intermediate frequency (IF) or baseband signal. The IF or baseband signal is sent to the RX processing circuitry 225, which generates a processed baseband signal by filtering, decoding, and/or digitizing the baseband or IF signal. The RX processing circuitry 225 transmits the processed baseband signal to the speaker 230 (such as for voice data) or to the processor 240 for further processing (such as for web browsing data).

The TX processing circuitry 215 receives analog or digital voice data from the microphone 220 or other outgoing baseband data (such as web data, e-mail, or interactive video game data) from the processor 240. The TX processing circuitry 215 encodes, multiplexes, and/or digitizes the outgoing baseband data to generate a processed baseband or IF signal. The RF transceiver 210 receives the outgoing processed baseband or IF signal from the TX processing circuitry 215 and up-converts the baseband or IF signal to an RF signal that is transmitted via the antenna 205.

The processor 240 can include one or more processors or other processors and execute the OS program 261 stored in the memory 260 in order to control the overall operation of the electronic device 101. For example, the processor 240 could control the reception of forward channel signals and the transmission of reverse channel signals by the RF transceiver 210, the RX processing circuitry 225, and the TX processing circuitry 215 in accordance with well-known principles. In some embodiments, the processor 240 includes at least one microprocessor or microcontroller.

The processor 240 is also capable of executing other processes and programs resident in the memory 260. The processor 240 can move data into or out of the memory 260 as required by an executing process. In some embodiments, the processor 240 is configured to execute the applications 262 based on the OS program 261 or in response to signals received from external devices or an operator. The processor can execute a resource management application 263 for monitoring system resources. The processor 240 is also coupled to the I/O interface 245, which provides the electronic device 101 with the ability to connect to other devices such as laptop computers, handheld computers and other accessories, for example, a virtual reality (VR) headset. The I/O interface 245 is the communication path between these accessories and the processor 240. The processor 240 can recognize accessories that are attached through the I/O interface 245, such as a VR headset connected to a USB port.

The processor 240 is also coupled to the input 250 and the display 255. The operator of the electronic device 101 can use the input 250 (e.g., keypad, touchscreen, button etc.) to enter data into the electronic device 101. The display 255 may be an LCD, LED, OLED, AMOLED, MEMS, electronic paper, or other display capable of rendering text and/or at least limited graphics, such as from web sites.

The memory 260 is coupled to the processor 240. Part of the memory 260 could include a random access memory (RAM), and another part of the memory 260 could include a Flash memory or other read-only memory (ROM).

The electronic device 101 further includes one or more sensors 265 that can meter a physical quantity or detect an activation state of the electronic device 101 and convert metered or detected information into an electrical signal. For example, the sensor 265 may include any of the various sensors 180 discussed above.

Although FIG. 2 illustrates one example of an electronic device 101, various changes may be made to FIG. 2. For example, various components in FIG. 2 could be combined, further subdivided, or omitted and additional components could be added according to particular needs. As a particular example, the processor 240 could be divided into multiple processors, such as one or more central processing units (CPUs) and one or more graphics processing units (GPUs). Also, while FIG. 2 illustrates the electronic device 101 configured as a mobile telephone or smart phone, electronic devices could be configured to operate as other types of mobile or stationary devices. In addition, as with computing and communication networks, electronic devices can come in a wide variety of configurations and FIG. 2 does not limit this disclosure to any particular electronic device.

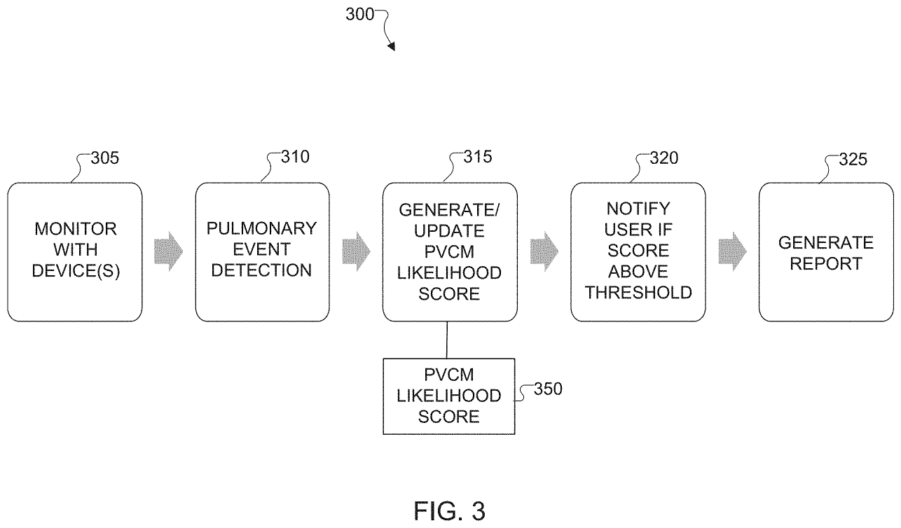

FIG. 3 illustrates an example process 300 for determining a likelihood of PVCM in a person in accordance with this disclosure. For ease of explanation, the process 300 is described as involving at least one electronic device (such as the electronic device 101 of FIG. 1). However, the process 300 could be used with any other suitable device or system without departing from the scope of this disclosure. Certain operations of the process 300 are presented here at a general level. Further details of the process 300 are provided below in conjunction with other figures.

As shown in FIG. 3, the process 300 includes operation 305, in which at least one electronic device monitors a user to track respiratory issues of the user (such as difficult breathing, coughing, and wheezing), context parameters related to the user and the environment around the user, medication intake by the user, and the like. This can include, for example, long-term monitoring of the user (e.g., over a period of days, weeks, or months) with wearable or mobile devices, such as a smart phone or smart watch, to extract PVCM-specific features from symptoms exhibited by the user.

At operation 310, the at least one electronic device detects and classifies pulmonary events associated with the user. This can include the electronic device detecting and classifying occurrences of breathing difficulty, coughs, or wheezes by the user. This can also include the electronic device detecting and classifying occurrences of medication intake, such as the user using an inhaler.

At operation 315, the at least one electronic device generates or updates a PVCM likelihood score 350 for the user based on the information collected or detected while monitoring the user. The electronic device generates the PVCM likelihood score 350 while taking into account the user's risk factors (e.g., age, previous asthma diagnosis, etc.), environmental factors (season, temperature, allergens in air), or both. The PVCM likelihood score 350 is determined based on a model that ranks the importance of the different features. The model can be dynamically updated over time based on observations from the sensors.

In some embodiments, the PVCM likelihood score 350 is represented by τ and is determined according to the following equation:

τ=αfₘ+βf_{d}+γf_{c}+δf_{w}+εfₐ (1)

where
fₘ is a value indicating a level of symptom improvement after medication intake,
α is a weight indicating an importance of the symptom improvement feature fₘ,
f_{d} is a value indicating respiration phase difficulty,
β is a weight indicating an importance of the respiration difficulty feature f_{d},
f_{c} is a value indicating a cough score,
γ is a weight indicating an importance of the cough score f_{c},
f_{w} is a value indicating a wheeze score,
δ is a weight indicating an importance of the wheeze score f_{w},
fₐ is a value indicating attack abruptness,
ε is a weight indicating an importance of the attack abruptness feature fₐ,
0≤α, β, γ, δ, ε, τ, fₘ, f_{d}, f_{c}, f_{w}, fₐ≤1, and

α+β+γ+δ+ε=1.

In some embodiments, the PVCM likelihood score τ can be rescaled to a range between 0 to 100%. The rescaling of τ can be performed based on observed values in a previously determined training dataset, and applying normalization such that the highest observed τ value is assigned a score of 100% (i.e., a score associated with severe PVCM patients) and the lowest observed τ value is assigned a score of 0% (i.e., a score associated with little likelihood of PVCM). Regression techniques can be used to formulate a relationship between the calculated τ value and the 0 to 100% scale. A higher score indicates a higher likelihood that the user has PVCM.

The rescaling of τ can be achieved by setting the weights α, β, γ, δ and ε to appropriate values. The initial values of the weights can be determined based on a combination of prior clinical knowledge on relative importance of these features, as well as a grid search on labeled training data collected prior to the deployment of the system. As an example, such training data can be collected from observation of patients in one or more research studies.

In one embodiment, the translation from user symptom observation data, obtained by the sensors, to the values for the features fₘ, f_{d}, f_{c}, f_{w} and fₐ can also be based on labeled training data. In one embodiment, these values can have a recall period of 1 week, meaning that the values for the features are updated based on symptoms and events of the past 1 week. In particular, the values observed in a prior training dataset can inform these scales.

For example, the number of wet coughs observed in the past 1 week for each patient in a prior research study can be noted. The maximum frequency of wet coughs in the dataset can be used to indicate a value of 1 for the f_{c} feature. All lower values of wet cough frequency can then be rescaled such that the f_{c} values observed in the dataset are all between 0 and 1 on a continuous scale.

Similarly, in a prior research study, each patient may have recorded audio of the patient's breathing, coughing, wheezing, etc., during respiratory attacks suffered by the patient. The patient may also have recorded the onset time and resolution time of each respiratory attack. The audio features surrounding these times can then be used to train a model to recognize the start and stop times for future attacks. Furthermore, the timing information of these attacks in the prior dataset can be used to create a scale for the fₐ parameter. For example, the lowest observed duration between start and stop times (thus indicating the most abrupt cough) can indicate a value of 1 for the fₐ feature, and the highest duration can indicate a value of 0. This can then be fit onto a regression model such that any future observed attack onset and resolution times can be translated to an fₐ value between 0 and 1 on a continuous scale.

The other features fₘ, f_{d}, and f_{w} can be defined in a similar fashion in this embodiment. For example, for the feature f_{w}, a value of 0 can indicate that the patient always wheezes on both exhalation and inhalation, while a value of 1 can indicate that the patient wheezes only on inhalation and never on exhalation. This reflects an observation that PVCM patients generally wheeze on inhalation but not on exhalation. For the feature f_{d}, a value of 1 can indicate that the patient experiences more difficulty on exhalation compared to inhalation, while a value of 0 can indicate that the patient exhibits the same level of difficulty (or less difficulty) for exhalation as compared to inhalation. For the feature fₘ, a value of 1 can indicate that the patient always exhibits a significant reduction in symptoms (e.g., reduced coughing, wheezing, etc.) relatively quickly after inhaler use, while a value of 0 can indicate that the patient exhibits no significant reduction in symptoms after inhaler use.

The determination of the PVCM likelihood score 350 can be most accurate when all of the user symptom observation data that contribute to the features fₘ, f_{d}, f_{c}, f_{w} and fₐ (i.e., symptom improvement after medication intake, respiration phase difficulty, cough scoring, wheeze tracking, and abruptness of attack) are available. When only a subset of the user symptom observation data is available, the operation 315 of determining the PVCM likelihood score 350 can include adjusting one or more of the weights α, β, γ, δ and ε to reflect availability of user symptom observation data. In some embodiments, the weights may be dynamically updated based on changes in the strength of the available feature sources and their frequency of occurrences.

As discussed above, prior knowledge of manifestation of symptoms according to research, as well as training data collected to reflect distribution of certain symptoms, can be used for a machine learning model to determine the weights α, β, γ, δ, and ε. For example, research has shown that approximately 80% of asthma patients exhibit sputum, while only 3% of PVCM patients do. This indicates that the wetness of a cough can be an important differentiator of asthma and PVCM patients, and thus the value for γ may be set to a higher value. An increase in the relative occurrence of wet coughs in a patient would then mean a lower value of f_{c} and hence a lower likelihood of PVCM.

Also, research has shown that approximately 90% of asthma patients show improvement after bronchodilator use, while only 10% of PVCM patients do. This suggests that the value of a should be set to a higher value. Therefore, if a patient exhibits a reduction in symptoms (e.g., cough, wheeze, etc.) relatively quickly after inhaler use, the fₘ feature will be higher and hence the likelihood of PVCM for that patient will increase. In addition, approximately 18% of PVCM patients show stridor (wheeze sounds during inspiratory phases), while only 6% of asthma patients do. This indicates that stridor is modestly indicative of PVCM, and the value of 6 may be set accordingly. Therefore, if a patient exhibits relatively more wheeze sounds during inspiration phases, the value of f_{w} will be higher and hence the PVCM score will increase. Based on these observations, the value of δ may be set higher relatively lower, such that an increase in f_{w} does not impact the PVCM score as much as the other more important features (i.e., fₘ and f_{c}).

Following the above rationale, one example implementation of the operation 315 may have the weights set up as: γ=0.4, α=0.4, and δ=0.2. For this implementation, the other two weights β and ε are set to zero, as associated symptoms have not been observed. Of course, these values are merely one example; other values that are higher or lower for each weight and feature are within the scope of this disclosure and may be used depending on the actual implementation.

If the PVCM likelihood score 350 is above a predetermined threshold, then at operation 320, the at least one electronic device notifies the user of the possibility that the user may have PVCM. This can include, for example, the electronic device presenting a message including the score on the display of the electronic device. This can also include the electronic device generating an audible alarm.

At operation 325, the at least one electronic device generates a report indicating one or more primary symptoms and contexts for PVCM attacks associated with the user. This can include, for example, the electronic device generating an electronic file containing the report or transmitting the report to a connected printer. The electronic device can also electronically transmit the report to a health care provider, such as by email. Upon a visit by the user to the health care provider, the report can be used by the provider to direct treatment.

The various functions and operations shown and described above with respect to FIG. 3 can be implemented in the at least one electronic device (which could include any of the electronic devices 101, 102, 104 or the server 106) in any suitable manner. For example, in some embodiments, at least some of the functions and operations can be implemented or supported using one or more software applications or other software instructions that are executed by the processor(s) 120, 240 of the electronic device(s). In other embodiments, at least some of the functions and operations can be implemented or supported using dedicated hardware components. In general, the functions and operations can be performed using any suitable hardware or any suitable combination of hardware and software/firmware instructions.

Although FIG. 3 illustrates one example of a process 300 for determining a likelihood of PVCM in a person, various changes may be made to FIG. 3. For example, various operations in FIG. 3 could overlap, occur in parallel, occur in a different order, or occur any number of times.

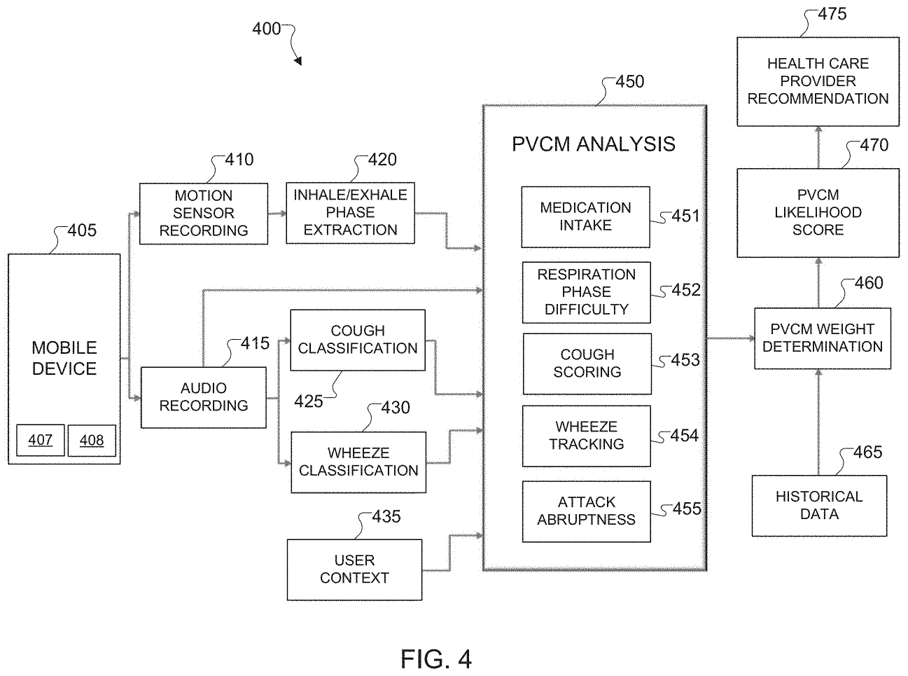

FIG. 4 illustrates an example system 400 for determining a likelihood of PVCM in a person in accordance with this disclosure. For ease of explanation, the system 400 is described as involving the process 300 shown in FIG. 3. However, the system 400 could be used with any other suitable process without departing from the scope of this disclosure.

As shown in FIG. 4, the system 400 includes a mobile device 405. The mobile device 405 is a mobile electronic device that is associated with (or used by) a user who may be a PVCM patient. The mobile device 405 may represent one of the electronic devices 101, 102, 104 of FIG. 1. For example, the mobile device 405 may be the user's smart phone, smart watch, or tablet. The mobile device 405 can include one or more motion sensors 407 that are capable of detecting and recording motion, acceleration, position, and the like, such as one or more of the sensors 180 of FIG. 1. The mobile device 405 can also include one or more audio sensors 408 that are capable of detecting and recording audio in the vicinity of the mobile device 405, such as the microphone 220 of FIG. 2.

To collect data associated with a likelihood of PVCM in the user, the mobile device 405 performs one or more motion recording functions 410 and one or more audio recording functions 415. These functions 410, 415 can represent operation 305 of FIG. 3. The motion recording function 410 can include detection and recording by the motion sensor 407 of motion associated with the user. Specifically, this can include the motion sensor 407 detecting motion of the user's chest while the user breathes, coughs, or wheezes. In some embodiments, the mobile device 405 is placed in contact with the user's chest for a short period of time (e.g., 30 to 60 seconds—a “spot check”), so that the motion sensor 407 can detect and record breathing movements through the chest wall. The spot checks can be scheduled to occur at regular intervals (e.g., 4 times a day), and the mobile device 405 may provide an audio or visual alert to the user when it is time to perform a spot check.

The motion recording function 410 can also include detecting motion of the user's hand toward the user's mouth when the user uses a respiratory inhaler. For example, when the mobile device 405 is a smart watch worn on the user's wrist, the motion sensor 407 can detect the motion of the user's hand while holding the inhaler. The motion recording function 410 can also include detecting movement of user's body as an indicator of physical activity. For example, when the user carries or wears the mobile device 405, the motion sensor 407 can detect that the user is walking, running, lying down, sitting, and the like.

The audio recording function 415 can include detection and recording by the audio sensor 408 of sounds associated with the user. Specifically, this can include the audio sensor 408 detecting sounds emanating from the user while the user breathes, coughs, or wheezes. The audio sensor 408 can detect the sounds through the chest wall while the mobile device 405 is placed in contact with the chest during a spot check. The audio sensor 408 can also detect respiratory sounds of the user when the mobile device 405 is merely in the vicinity of (but not necessarily in contact with) the user. The audio recording function 415 can also include the audio sensor 408 detecting the “puff” of a respiratory inhaler used by the user.

The motion recording function 410 and audio recording function 415 can be performed continuously or at regular intervals over a predetermined period of time (e.g., twenty-four hours). In general, the motion recording function 410 and audio recording function 415 are passive and automatic, requiring little or no intervention by the user. As the motion and audio information is collected by the sensors 407-408, the raw motion and sound data is recorded and stored in a memory with timestamps, so that movements and sounds can later be correlated with time and event information. In some embodiments, the mobile device 405 can transmit the raw motion and sound data to another electronic device (e.g., the server 106) for storage or processing.

Using the motion and sound data collected by the functions 410, 415, the mobile device 405 performs an inhale/exhale phase extraction function 420, a cough classification function 425, and a wheeze classification function 430 for extraction and classification of the data. The functions 420-430 execute sound and motion detection algorithms to analyze the raw sound and motion data, parse out particular sound and motion features (e.g., coughing, wheezing, medication intake, and the like) and determine characteristics of each feature (time of occurrence, duration, wetness, severity, etc.). The functions 420-430 can represent operation 310 of FIG. 3.

During the inhale/exhale phase extraction function 420, the mobile device 405 separates motion and sound data associated with user breathing from data associated with other activities (e.g., talking, background noises, physical movements, and the like). The mobile device 405 then classifies the breathing data as being related to an inhalation or an exhalation. During the cough classification function 425, the mobile device 405 parses out coughing sounds and movements and determines characteristics of each cough (time of occurrence, duration, wetness, severity, etc.). During the wheeze classification function 430, the mobile device 405 parses out wheezing sounds and movements and determines characteristics of each wheeze (time of occurrence, duration, wetness, severity, etc.).

The mobile device 405 also receives or obtains user context information 435. The user context information 435 is information associated with the user that can provide context for the occurrence of PVCM symptoms (or lack of symptoms). The user context information 435 can include environmental conditions (e.g., current time, air temperature, air pressure, humidity, location (geographical or indoor vs. outdoor), presence of pollutants or allergens in air, etc.), demographic and medical information of the user (e.g., age, gender, ethnicity, weight, body temperature, blood pressure, history of ailments, etc.), current or historical activity level of the user (e.g., standing, sitting, reclining, walking, running, sleeping, eating, etc.) and the like. Demographic information can be entered by the user or automatically obtained from another source (e.g., an electronic medical file) during an initialization operation of the system 400. Other contextual information can be obtained in real time from one or more sensors of the mobile device 405, such as a GPS sensor.

Once the mobile device 405 performs the functions 420-430 and receives the user context information 435, the mobile device 405 performs a PVCM analysis function 450. The PVCM analysis function 450 comprises multiple functions, including a medication intake function 451, a respiration phase difficulty function 452, a cough scoring function 453, a wheeze tracking function 454, and an attack abruptness function 455. Each function 451-455 results in a score corresponding to one of the features fₘ, f_{d}, f_{c}, f_{w}, and fₐ described above with respect to FIG. 3. While FIG. 4 shows the PVCM analysis function 450 comprised of five functions 451-455, in some embodiments, one or more of the functions 451-455 may not be performed, depending on the sound or motion data available. In addition, the functions 451-455 could be performed in any suitable order.

The medication intake function 451 analyzes the sound or motion data to determine sounds or motions in the collected data that are associated with the use of a respiratory inhaler (e.g., the “puff” sound of the inhaler dispensing the medication). The medication intake function 451 correlates the sounds with timestamp data to determine when the user used the inhaler. The medication intake function 451 also correlates the time of inhaler use with the user's improved respiratory symptoms, if any. In some embodiments, the medication intake function 451 can use one or more template matching techniques, such as dynamic time warping, to correlate the observed inhaler use and symptoms with their respective times. The medication intake function 451 can determine improved respiratory symptoms by determining an improvement in breathing, coughing, or wheezing over time based on the information classified by the functions 420-430. In an asthmatic patient, symptoms will usually improve quickly after use of an inhaler. Patients with PVCM typically see little or no improvement after use of an inhaler. Based on the determined improved respiratory symptoms, the medication intake function 451 assigns a value between 0 and 1 to the feature fₘ indicating a level of improvement after medication intake.

The respiration phase difficulty function 452 analyzes the sound or motion data to determine if the user exhibits more difficulty in inhalation than during exhalation, which is a symptom of PVCM. In contrast, an asthma patient usually exhibits difficulty with both inhalation and exhalation. For example, the respiration phase difficulty function 452 can examine differences between the inhalation data and the exhalation data classified by the inhale/exhale phase extraction function 420. Based on the determined differences in difficulty between inhalation and exhalation, the respiration phase difficulty function 452 assigns a value between 0 and 1 to the feature f_{d} indicating respiration phase difficulty.

The cough scoring function 453 analyzes the sound or motion data of coughs classified by the cough classification function 425 to analyze trends of dry versus wet coughs over time. The cough scoring function 453 can also use the user context information 435 to take into account confounding factors for wetness of certain coughs, such as a high concentration of allergens in the air at the time of the cough, or an indication of intense physical activity by the user immediately prior to the cough. Based on the determined trends of wet and dry coughs, the cough scoring function 453 assigns a value between 0 and 1 to the feature f_{c} indicating a cough score, which is associated with cough wetness level.

The wheeze tracking function 454 analyzes the sound or motion data of wheezes classified by the wheeze classification function 430 to determine how many of the wheezes are actually stridor. The wheeze tracking function 454 then determines whether or not there is a predominance of stridor over general wheezes, which is a symptom of PVCM. Based on the proportions of stridor to general wheezes, the wheeze tracking function 454 assigns a value between 0 and 1 to the feature f_{w} indicating a wheeze score.

The attack abruptness function 455 analyzes the sound or motion data and timestamp data to determine the start time, end time, and duration of each respiratory attack suffered by the user. Each cough and its abruptness characteristics can be correlated to trend data to determine if the cough is more or less sudden or abrupt compared to other coughs, which can be indicative of a PVCM attack or an asthma attack. Based on this determination, the attack abruptness function 455 assigns a value between 0 and 1 to the feature fₐ indicating attack abruptness.

The mobile device 405 also performs a PVCM weight determination function 460 to determine the PVCM weights α, β, γ, δ, and ε that correspond to the features fₘ, f_{d}, f_{c}, f_{w} and fₐ as described above with respect to FIG. 3. The weights are used to weight and rank the features based on their predicted importance in determining the likelihood of PVCM. The mobile device 405 performs the PVCM weight determination function 460 as described in FIG. 3, balancing clinical importance with respect to PVCM and characteristics of occurrence in the audio and motion recordings. The PVCM weights can be updated dynamically over time, based on new training data (which may originate with the user or with a larger population group). Historical data 465 can also be considered in determining the PVCM weights. The historical data 465 can include training data of the patient or of other populations, especially those with demographics similar to the patient. For example, if historical data indicates that the patient never (or almost never) coughs, then it might be more accurate to reduce the importance of the cough score f_{c} by lowering the value of γ.

Using the PVCM features determined by the PVCM analysis function 450 and the PVCM weights determined by the PVCM weight determination function 460, the mobile device 405 generates a PVCM likelihood score 470, which indicates how likely it is that the user has PVCM versus asthma. The PVCM analysis function 450 can represent operation 315 of FIG. 3, and the PVCM likelihood score 470 can represent the PVCM likelihood score 350. That is, the PVCM likelihood score 470 can be determined according to Equation (1) as discussed above. If the PVCM likelihood score 470 is above a predetermined threshold, then the mobile device 405 can notify the user of the possibility that the user may have PVCM. For example, the mobile device 405 can present a message including the PVCM likelihood score 470 on the display of the mobile device 405. As another example, the mobile device 405 can generate an audible alarm or tone indicating the condition.

If the generated PVCM likelihood score 470 is high enough to suggest a likelihood of PVCM, the mobile device 405 can generate a health care provider recommendation 475 and provide the recommendation 475 to a health care provider treating the user. The recommendation 475 can include the PVCM likelihood score 470 and any specific respiratory information of the user collected by the mobile device 405, such as one or more likely triggers for PVCM attacks, which can aid laryngoscopy. The recommendation 475 can include information presented on the display of the mobile device 475 for the health care provider to view, an email or other electronic message transmitted (e.g., over a network, such as the Internet) to an electronic device of the health care provider, or any other suitable method for communicating the score and recommendation.

In some embodiments, the system 400 can operate over a long period of time (e.g., a few weeks to several months or longer), with the mobile device 405 continually or regularly monitoring the user and obtaining new or updated motion and audio data from the user. As new data is collected, the PVCM likelihood score 470 can be generated multiple times, and each new score 470 can be based (at least in part) on user symptom data collected over a longer period of time. Long-term monitoring can make it more likely to capture differences in symptoms between PVCM and asthma, as PVCM symptoms can be very subtle and unlikely to manifest clearly in a one-time measurement. In some applications, a preferred period for long term monitoring can be from one month to six months depending on the frequency of presentation of symptoms. However, it could be shorter than one month or longer than six months, depending on the application or patient. In general, the accuracy of at least some of the components of the system 400 can improve as more respiratory data is obtained to fine-tune the parameters.

The PVCM likelihood score 470 and the other respiratory data obtained or generated by the system 400 can complement laryngoscopy by providing data over longer periods of time (which laryngoscopy cannot). In particular, by analyzing patterns of symptoms over time, the system 400 can predict the possible occurrence of upcoming instances of symptoms in the user, and provide information to the health care provider indicating a time when the symptoms of PVCM may occur. This can be very helpful in scheduling a laryngoscopy, since the procedure is most successful when a PVCM attack occurs while the tube is inserted. The system 400 can also improve detection rates of the disease since large numbers of patients will never undergo laryngoscopy and hence may go misdiagnosed.

Although FIG. 4 illustrates one example of a system 400 for determining a likelihood of PVCM in a person, various changes may be made to FIG. 4. For example, the system 400 could be implemented in any suitable manner, such as entirely within the mobile device 405 or using a combination of devices. For instance, the mobile device 405 could collect the audio and motion data and provide the data to a server 106, which could then perform some of the operations of FIG. 4, including the PVCM analysis function 450 and the generation of the PVCM likelihood score 470. Results of the processing could then be made available to the mobile device 405 and/or one or more other devices (such as the electronic device 102 or 104). In general, computing and communication systems come in a wide variety of configurations, and FIG. 4 does not limit the scope of this disclosure to any particular configuration. Some examples of other configurations will now be described.

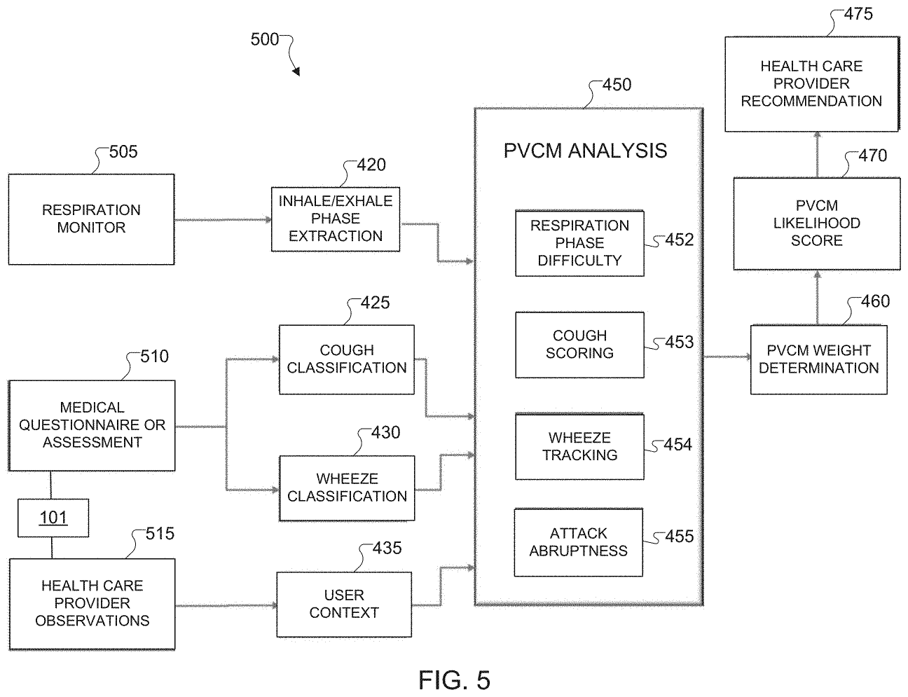

FIG. 5 illustrates another example system 500 for determining a likelihood of PVCM in a person in accordance with this disclosure. For ease of explanation, the system 500 is described as involving the use of the process 300 shown in FIG. 3. However, the system 500 could be used with any other suitable process without departing from the scope of this disclosure.

As shown in FIG. 5, the system 500 includes a number of components that are the same as or similar to corresponding components of the system 400. In contrast to the system 400 (in which the mobile device 405 automatically collects data from the user via one or more sensors), the system 500 includes collection of data from multiple sources, including electronic devices other than a user's mobile device. For example, the system 500 includes a respiration monitor 505, a medical questionnaire or assessment 510, and health care provider observations 515.

The respiration monitor 505 can be in a medical office or hospital and can monitor the respiration of the user when the user visits the medical office. This may include a scenario where the user has an acute respiratory attack and visits an emergency room. In some embodiments, the respiration monitor 505 is a dedicated, medical grade equipment as known in the art. The respiration monitor 505 can collect inhalation and exhalation data that can be received as input for the inhale/exhale phase extraction function 420. Although not explicitly shown in FIG. 5, the respiration monitor 505 could also represent two or more respiration monitors, or a respiration monitor and one or more other types of medical equipment for monitoring patient symptoms.

The medical questionnaire 510 is a collection of questions and answers or another type of medical assessment to obtain information related to respiratory issues of the user (such as times and severity of coughs or wheezes) when the user visits the medical office. The medical questionnaire 510 can include electronically generated questions shown on a display of an electronic device 101, such as a tablet or computer in the medical office. The answers to the medical questions can be input on the electronic device 101, either by the user or by a medical professional. The answers can be received as input for the cough classification function 425, the wheeze classification function 430, or both.

The health care provider observations 515 include information related to the overall health of the user that are obtained by the health care provider when the user visits the medical office. The information can include general health assessment information, such as age, gender, weight, blood pressure, body temperature, blood test results, and the like. The health care provider observations 515 can include information electronically collected at an electronic device 101, such as a tablet or computer in the medical office. The health care provider observations 515 can be received as input for the user context information 435.

Once the data 420-435 is collected or input, the system 500 can perform the PVCM analysis function 450 and calculated the PVCM likelihood score 470. If new or updated data is received over time (such as during a longer hospital stay), the system 500 can perform the PVCM analysis function 450 again and recalculate the PVCM likelihood score 470 using the new or updated data. Of course, in a short visit to a medical office or hospital, long term patient data (e.g., data collected over a period of weeks) may not be available. However, if the data is made available to a server, the same PVCM likelihood estimation can be run on the shorter time scale of data (e.g., one day's worth of data) to provide a score for the user.

Although FIG. 5 illustrates one example of a system 500 for determining a likelihood of PVCM in a person, various changes may be made to FIG. 5. For example, the system 500 could include any number of each component in any suitable arrangement. In general, computing and communication systems come in a wide variety of configurations, and FIG. 5 does not limit the scope of this disclosure to any particular configuration.

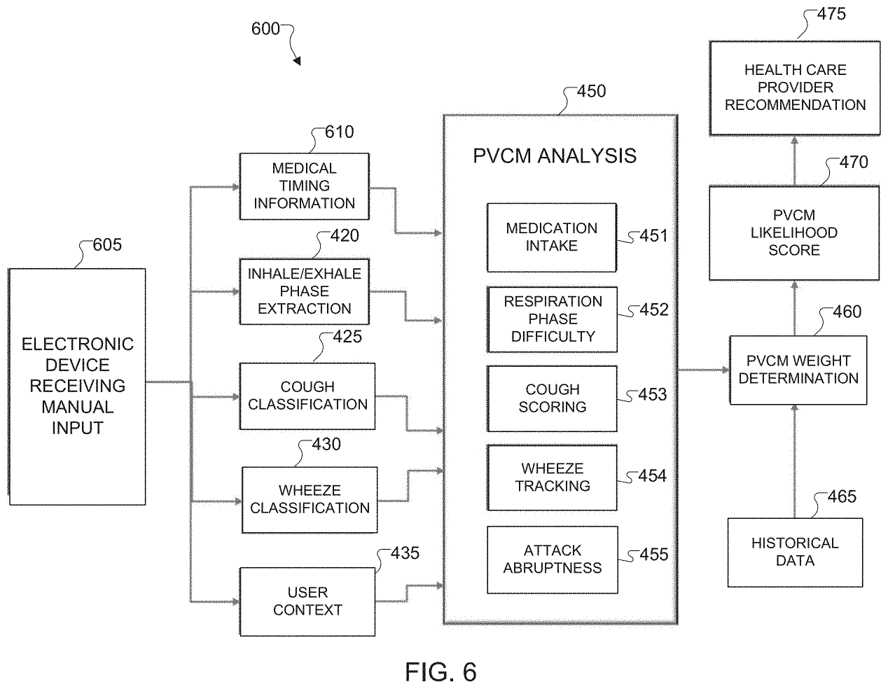

FIG. 6 illustrates yet another example system 600 for determining a likelihood of PVCM in a person in accordance with this disclosure. For ease of explanation, the system 600 is described as involving the process 300 shown in FIG. 3. However, the system 600 could be used with any other suitable process without departing from the scope of this disclosure.

As shown in FIG. 6, the system 600 includes a number of components that are the same as or similar to corresponding components of the system 400. In contrast to the system 400 (in which the mobile device 405 automatically collects data from the user via one or more sensors), the system 600 operates using user inputs to an electronic device 605 that are provided manually by the user or a user representative over time. Here, the electronic device 605 could be a mobile device or a desktop computer, and the information could be manually entered by the user into an a computer application, a mobile “app”, or a website. For example, the user can manually input medication timing information 610 on the electronic device 605. This can include information related to when the user has taken respiratory medication and what symptoms or change of symptoms (if any) that the user has noticed following the medication. As another example, the user can manually input the frequency of wet and dry coughs, occurrences of wheezing and stridor, and the like. This information can be organized and classified by the electronic device 605 while performing the inhale/exhale phase extraction function 420, the cough classification function 425, and the wheeze classification function 430. In one example scenario, a parent inputs data on a regular basis (e.g., daily or at another time interval) about their child based on manual observation of the child. Performing manual entry may be preferable to having the child carry a mobile phone or wear a watch continuously to record the sensor data, especially when the child is very young or has difficulty keeping up with a phone or watch.

Once the data 420-435, 610 is collected or input by the user, the system 600 can perform the PVCM analysis function 450 and calculate the PVCM likelihood score 470. As new or updated data is manually input over time, the system 600 can perform the PVCM analysis function 450 again and recalculate the PVCM likelihood score 470 using the new or updated data.

Although FIG. 6 illustrates one example of a system 600 for determining a likelihood of PVCM in a person, various changes may be made to FIG. 6. For example, the system 600 could include any number of each component in any suitable arrangement. In general, computing and communication systems come in a wide variety of configurations, and FIG. 6 does not limit the scope of this disclosure to any particular configuration.

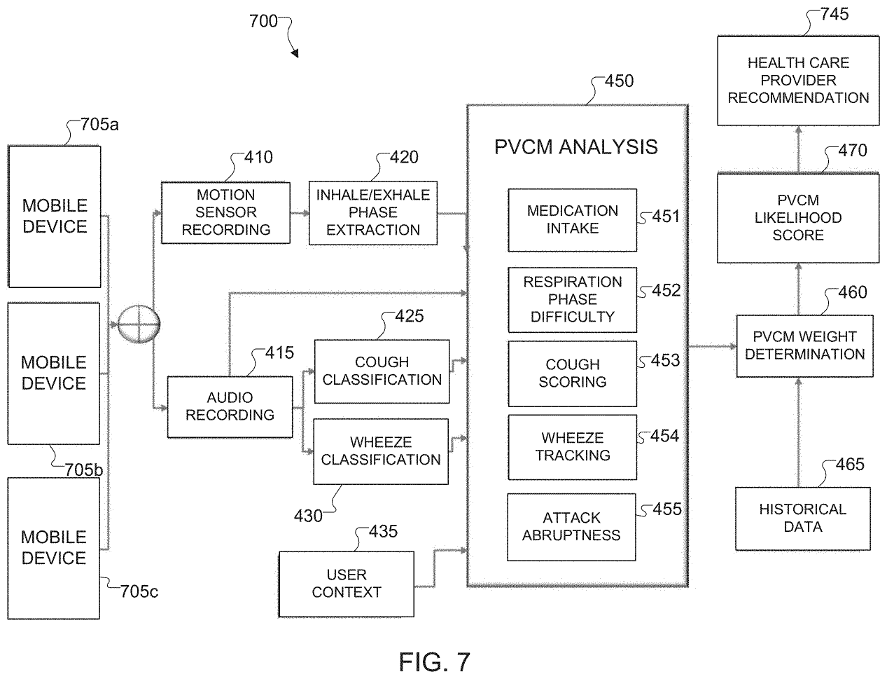

FIG. 7 illustrates still another example system 700 for determining a likelihood of PVCM in a person in accordance with this disclosure. For ease of explanation, the system 700 is described as involving the process 300 shown in FIG. 3. However, the system 700 could be used with any other suitable process without departing from the scope of this disclosure.

As shown in FIG. 7, the system 700 includes a number of components that are the same as or similar to corresponding components of the system 400. In contrast to the system 400 (which depicts one mobile device 405), the system 700 includes multiple mobile devices 705a-705c. In this example, the mobile device 705a is a smart phone, the mobile device 705b is a smart watch, and the mobile device 705c can be another smart phone, another smart watch, a mobile medical device, an electronic bracelet, or the like. Of course, this is merely one example, and other types and combinations of mobile devices are possible, including embodiments with only two mobile devices or more than three mobile devices.

When the user has access to multiple mobile devices 705a-705c, the mobile devices 705a-705c can operate together in the system 700 to collect, process, or store data associated with determining the likelihood of PVCM in the user. The mobile devices 705a-705c can operate simultaneously or at different times to collect, process, or store the data. For example, the mobile device 705b can detect and record motion information associated with the user's physical activity, while the mobile device 705a can detect and record audio data associated with the user's breathing.

The information from the multiple mobile devices 705a-705c can be collected at one mobile device 705a-705c before proceeding to the PVCM analysis function 450. For example, when the mobile device 705a performs the PVCM analysis function 450, the mobile device 705a can first receive some or all of the information used in the PVCM analysis function 450 from the other mobile devices 705b-705c. In some embodiments, the information can be sent to the mobile device 705a over a wireless network, such as a BLUETOOTH network. Communication between the mobile devices 705b-705c can be continuous in real time, in response to discrete data transmission requests, or according to any other suitable schedule.

In some scenarios, the use of multiple mobile devices 705a-705c can provide data from different types of sensors or from different locations or perspectives, which can result in a richer set of data and can result in a more accurate PVCM likelihood score 470. If any of the mobile devices 705a-705c are not present, working, or communicating at any given time, the remaining device(s) can operate in the system 700. If only one of the mobile devices 705a-705c is in operation at a given time, then the system 700 can operate similar to the system 400 of FIG. 4.

Although FIG. 7 illustrates one example of a system 700 for determining a likelihood of PVCM in a person, various changes may be made to FIG. 7. For example, the system 700 could include any number of each component in any suitable arrangement. In general, computing and communication systems come in a wide variety of configurations, and FIG. 7 does not limit the scope of this disclosure to any particular configuration.

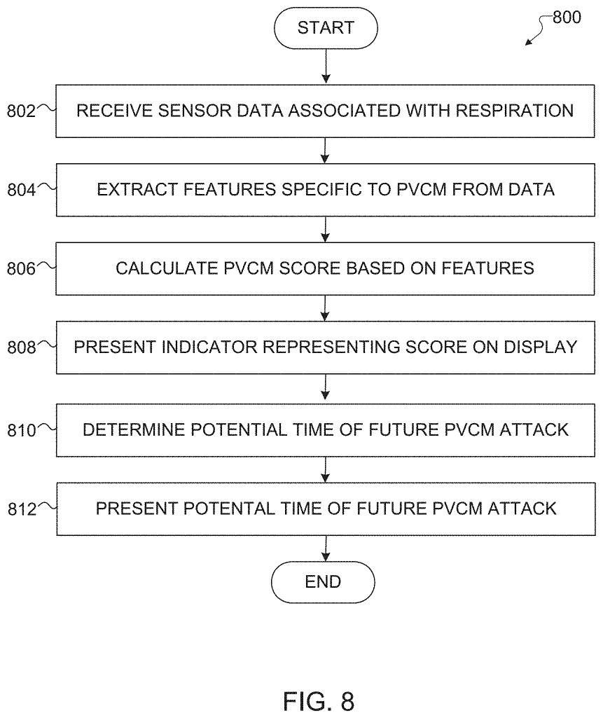

FIG. 8 illustrates an example method 800 for determining a likelihood of PVCM in a person in accordance with this disclosure. For ease of explanation, the method 800 shown in FIG. 8 is described as involving the use of the mobile device 405 of FIG. 4. However, the method 800 shown in FIG. 8 could be used with any other suitable electronic device and in any suitable system.

At operation 802, the mobile device 405 receives sensor data associated with respiration of a user. This can include, for example, the mobile device 405 receiving motion data or sound data from one or more sensors of the electronic device, such as in the motion recording functions 410 and one or more audio recording functions 415 of FIG. 4.

At operation 804, the mobile device 405 extracts features specific to PVCM from the sensor data. In some embodiments, the extracted features include a cough wetness level and a respiration phase difficulty level. This can include, for example, the mobile device 405 determining a cough score and a respiration phase difficulty feature based on the extracted features. In some embodiments, the mobile device 405 can also determine a symptom improvement feature, a wheeze score, or an attack abruptness feature.

At operation 806, the mobile device 405 calculates a PVCM score based on the extracted features using a predetermined model. The predetermined model allocates weights to the extracted features based on respective importance to PVCM determination. This can include, for example, the mobile device 405 calculating the PVCM likelihood score 470 according to equation (1).

At operation 808, the mobile device 405 presents an indicator on a display of the electronic device 101 for use by the user or a health care provider. The indicator represents the PVCM score. This can include, for example, the mobile device 405 presenting the PVCM likelihood score 470, and can also include a recommendation for treatment.

At operation 810, the mobile device 405 determines a potential time of a future PVCM attack by the user based on an analysis of patterns of the extracted features over a prior period of time. This can include, for example, the mobile device 405 analyzing respiratory symptom data collected over time by the sensors 407-408, determining one or more temporal patterns of PVCM symptoms that occur in the user, and then extrapolating the patterns to predict a potential time or times that one or more future PVCM attacks are likely to occur.

At operation 812, the mobile device 405 presents the potential time of the future PVCM attack on the display of the first electronic device, so that a medical procedure can be scheduled at the potential time. This can include, for example, the mobile device 405 presenting the predicted time to the health care provider so that a laryngoscopy can be scheduled to coincide with the predicted time.

Although FIG. 8 illustrates one example of a method 800 for determining a likelihood of PVCM in a person, various changes can be made to FIG. 8. For example, various steps in FIG. 8 could overlap, occur in parallel, occur serially, occur in a different order, or occur any number of times. Also, the steps of the method 800 could be implemented in any suitable manner, such as entirely within the mobile device 405 or using a combination of devices. For instance, the mobile device 405 could collect data and provide the data to a server 106, which could then process the data and generate any suitable output.

Although this disclosure has been described with reference to various example embodiments, various changes and modifications may be suggested to one skilled in the art. It is intended that this disclosure encompass such changes and modifications as fall within the scope of the appended claims.

What is claimed is:

1. A method, comprising:
receiving sensor data associated with respiration of a user at a first electronic device;
extracting features specific to paradoxical vocal cord motion (PVCM) from the sensor data, the extracted features comprising a cough wetness level and a respiration phase difficulty level;
calculating a PVCM score based on the extracted features using a predetermined model, wherein the predetermined model allocates weights to the extracted features based on respective importance to PVCM determination; and
presenting an indicator on a display of the first electronic device for use by the user or a medical provider, the indicator representing the PVCM score.

2. The method of claim 1, further comprising:
determining a potential time of a future PVCM attack by the user based on an analysis of patterns of the extracted features over a prior period of time; and
presenting the potential time of the future PVCM attack on the display of the first electronic device, so that a medical procedure can be scheduled at the potential time.

3. The method of claim 1, wherein the sensor data includes audio data associated with sounds generated by a body of the user.

4. The method of claim 3, wherein the audio data is obtained by the first electronic device or a second electronic device.

5. The method of claim 3, wherein at least some of the audio data is obtained by a medical device of a medical care office or hospital and transmitted to the first electronic device.

6. The method of claim 1, wherein the extracted features further comprise at least one of: wheeze condition, abruptness of respiratory attack, or asthma medication intake timing.

7. The method of claim 1, further comprising:
identifying one or more features of the extracted features as PVCM triggering features based on context associated with the one or more features, wherein the context includes one or more of: time of occurrence of the one or more features or activity level at the time of occurrence.

8. The method of claim 1, further comprising:
processing the sensor data to perform at least one of:
detecting respiration phases of inhaling and exhaling,
identifying a cough, or
recognizing a wheeze associated with the respiration phases.

9. An electronic device comprising:
a display;
a processor configured to:
receive sensor data associated with respiration of a user;
extract features specific to paradoxical vocal cord motion (PVCM) from the sensor data, the extracted features comprising a cough wetness level and a respiration phase difficulty level;
calculate a PVCM score based on the extracted features using a predetermined model, wherein the predetermined model allocates weights to the extracted features based on respective importance to PVCM determination; and
present an indicator on the display for use by the user or a medical provider, the indicator representing the PVCM score.

10. The electronic device of claim 9, wherein the memory further stores instructions executable by the processor to:
determine a potential time of a future PVCM attack by the user based on an analysis of patterns of the extracted features over a prior period of time; and
present the potential time of the future PVCM attack on the display, so that a medical procedure can be scheduled at the potential time.

11. The electronic device of claim 9, wherein the sensor data includes audio data associated with sounds generated by a body of the user.

12. The electronic device of claim 11, wherein the audio data is obtained by the electronic device or a second electronic device.

13. The electronic device of claim 11, wherein at least some of the audio data is obtained by a medical device of a medical care office or hospital and transmitted to the electronic device.

14. The electronic device of claim 9, wherein the extracted features further comprise at least one of: wheeze condition, abruptness of respiratory attack, or asthma medication intake timing.

15. The electronic device of claim 9, wherein the memory further stores instructions executable by the processor to:
identify one or more features of the extracted features as PVCM triggering features based on context associated with the one or more features, wherein the context includes one or more of: time of occurrence of the one or more features or activity level at the time of occurrence.

16. The electronic device of claim 9, wherein the memory further stores instructions executable by the processor to:
process the sensor data to perform at least one of:
detect respiration phases of inhaling and exhaling,
identify a cough, or
recognize a wheeze associated with the respiration phases.

17. A non-transitory computer readable medium containing computer readable program code that, when executed, causes at least one processor to:
receive sensor data associated with respiration of a user;
extract features specific to paradoxical vocal cord motion (PVCM) from the sensor data, the extracted features comprising a cough wetness level and a respiration phase difficulty level;
calculate a PVCM score based on the extracted features using a predetermined model, wherein the predetermined model allocates weights to the extracted features based on respective importance to PVCM determination; and
present an indicator on a display for use by the user or a medical provider, the indicator representing the PVCM score.

18. The non-transitory computer readable medium of claim 17, wherein the computer readable program code, when executed, further causes at least one processor to:
determine a potential time of a future PVCM attack by the user based on an analysis of patterns of the extracted features over a prior period of time; and
present the potential time of the future PVCM attack on the display, so that a medical procedure can be scheduled at the potential time.

19. The non-transitory computer readable medium of claim 17, wherein the sensor data includes audio data associated with sounds generated by a body of the user.

20. The non-transitory computer readable medium of claim 19, wherein the audio data is obtained by an electronic device comprising the non-transitory computer readable medium or a second electronic device.
